(19) United States
(12) Patent Application Publication
(10) Pub. No.: US 2013/0096469 A1
(43) Pub. Date: Apr. 18, 2013

(54) ULTRASOUND NEUROMODULATION TREATMENT OF OBESITY AND EATING DISORDERS

(71) Applicant: David J. Mishelevich, Playa del Rey, CA (US)

(72) Inventor: David J. Mishelevich, Playa del Rey, CA (US)

(21) Appl. No.: 13/539,308

(22) Filed: Jun. 30, 2012

Related U.S. Application Data
(60) Provisional application No. 61/547,679, filed on Oct. 15, 2011.

(51) Int. Cl. A61N 7/00

(52) U.S. Cl. 601/2

(57) ABSTRACT

Disclosed are methods and systems for non-invasive neuromodulation using ultrasound to treat obesity and eating disorders. The neuromodulation can produce acute or long-term effects. The latter occur through Long-Term Depression (LTD) and Long-Term Potentiation (LTP) via training. Included is control of direction of the energy emission, intensity, frequency, pulse duration, pulse pattern, and phase/intensity relationships to targeting and accomplishing up regulation and/or down regulation.

### CROSS REFERENCE TO RELATED APPLICATIONS

This patent application claims priority to Provisional Patent Application No. 61/547,679 filed Oct. 15, 2011, entitled “ULTRASOUND NEUROMODULATION TREATMENT OF EATING DISORDERS.”

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity for the treatment of a medical condition.

### BACKGROUND OF THE INVENTION

It has been demonstrated that focused ultrasound directed at neural structures can stimulate those structures. If neural activity is increased or excited, the neural structure is up regulated; if neural activated is decreased or inhibited, the neural structure is down regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22 (2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3 (10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, William, James P., PCT/US2009/050560, WO 2010/009141, published 2011 Jan. 21).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 500 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. patent application Ser. No. 12/263,026 published as U.S. 2009/0112133 A1, Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 Feb; 24 (2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

Because of the utility of ultrasound in the neuromodulation of deep-brain structures, it would be both logical and desirable to apply it to the treatment of obesity and eating disorders.

### SUMMARY OF THE INVENTION

It is the purpose of this invention to provide methods and systems for non-invasive neuromodulation using ultrasound to treat obesity and eating disorders. Eating disorders include, but are not limited to, Anorexia Nervosa and Bulimia Nervosa. Such neuromodulation can produce acute effects or Long-Term Potentiation (LTP) or Long-Term Depression (LTD). Included is control of direction of the energy emission, intensity, frequency (carrier frequency and/or neuromodulation frequency), pulse duration, pulse pattern, and phase/intensity relationships to targeting and accomplishing up-regulation and/or down-regulation. Use of ancillary monitoring or imaging to provide feedback is optional. In embodiments where concurrent imaging is performed, the device of the invention is constructed of non-ferrous material.

Multiple targets can be neuromodulated singly or in groups to treat obesity and eating disorders. To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS).

For treatment of obesity, primary neural targets are the Orbito-Frontal Cortex (OFC) (Cohen, J I, Yates K F, Duong, F, and A Convit, “Obesity, Orbitofrontal Structure and Functional Are Associated with Food Choice: a Cross-Sectional Study,” BMJ Open 2011; 1:e000175. doi:10.1136/bmjopen-2011-000175) that is to be up regulated, the Ventromedial Hypothalamus (VMH) (Melega, W P, Lacan, G, Gorfulho, A A, Behnke, E J and A A De Salles, “Hypothalamic deep brain stimulation reduces weight gain in an obesity-animal model,” PLoS One. 2012; 7 (1):e30672. Epub 2012 Jan. 25) that is to be up regulated bilaterally, the Lateral Hypothalamus (LH) (Halpern C H, Wolf J A, Bale T L, Stunkard A J, Danish S F, Grossman M, Jaggi J L, Grady M S, and G H Baltuch, “Deep brain stimulation in the treatment of obesity,” J Neurosurg. 2008 Oct; 109 (4):625-34) that is to be down regulated and the Nucleus Accumbens (Berthoud, H-R, Lenard, N R, and A C Shim, “Food reward, hyperphagia, and obesity,” Amer. J of Physiol., Regulatory, Integrative, and Comparative Physiology, June 1, 2011 vol. 300 no. 6 R1266-R1277).

Targets for treating the eating disorder Anorexia Nervosa have been identified such the Anterior Cingulate Cortex (ACC) and the Pre-Frontal Cortex (PFC). As to eating disorders, in patients with Anorexia Nervosa, the volume of the Anterior Cingulate Cortex (ACC) is decreased (Mühlau M, Gaser C, Ilg R, Conrad B, Leibl C, Cebulla M H, Backmund H, Gerlinghoff M, Lommer P, Schnebel A, Wohlschläger A M, Zimmer C, and S Nunnemann, “Gray matter decrease of the anterior cingulate cortex in anorexia nervosa,” Am J Psychiatry. 2007 Dec; 164 (12):1850-7). Regional cerebral blood flow have shown that the Pre-Frontal Cortex (PFC) is involved (Matsumoto R, Kitabayashi Y, Narumoto J, Wada Y, Okamoto A, Ushijima Y, Yokoyama C, Yamashita T, Takahashi H, Yasuno F, Suhara T, and K Fukui, “Regional cerebral blood flow changes associated with interoceptive awareness in the recovery process of anorexia nervosa,” Prog Neuropsychopharmacol Biol Psychiatry. 2006 Sep 30; 30 (7):1265-70. Epub 2006 Jun 14). Other targets identified in the study were the Anterior Cingulate Cortex (ACC) and the Posterior Cingulate Cortex (PCC). Among the targets identified in patients with Bulimia are the Anterior Cingulate Cortex (ACC), and Dorsal Anterior Cingulate Gyms (DACG) and the Caudate Nucleus (Marsh, R, Steinglass, J, Gerber, A J, O'Leary, K G, Wang, Z, Murphy, D, Walsh, B T, and B S Peterson, “Deficient Activity in the Neural Systems That Mediate Self-regulatory Control in Bulimia Nervosa,” Arch Gen Psychiatry. 2009; 66 (1):51-63). Other targets related to Bulimia are superior temporal gyrus/insula (relative deactivation relative to normal), the Pre-Frontal Cortex, and the Caudate Nucleus (Brooks, S J, O'Daly, O G, Uher, R, Friederich, H-C, Giampietro, V, Brammer, M, Williams, S C R, Schiöth, H B, Treasure, J, and I C Campbell, “Differential Neural Responses to Food Images in Women with Bulimia versus Anorexia Nervosa,” PLoS One. 2011; 6 (7): e22259, Published online 2011 July 20. doi: 10.1371/journal.pone.0022259).

Targets depend on specific patients and relationships among the targets. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target.

### BRIEF DESCRIPTION OF THE DRAWINGS

FIG. 1 shows ultrasonic-transducer targeting of the Orbito-Frontal Cortex (OFC), Ventromedial Hypothalamus (VMH), the Lateral Hypothalamus (LH), and the Nucleus Accumbens (NAc) for the treatment of obesity.

FIG. 2 shows ultrasonic-transducer targeting of Pre-Frontal Cortex (PFC) and Anterior Cingulate Cortex for the treatment of Anorexia Nervosa.

FIG. 3 shows ultrasound-transducer targeting of the Caudate Nucleus, the Dorsal Anterior Cingulate Gyms (DACG), Pre-Frontal Cortex (PFC), Anterior Cingulate Cortex (ACC), the Insula, and Temporal Lobe for the treatment of Bulimia.

FIG. 4 shows a block diagram of the control circuit.

### DETAILED DESCRIPTION OF THE INVENTION

It is the purpose of this invention to provide methods and systems and methods for neuromodulation of deep-brain targets using ultrasound to treat obesity and eating disorders. Such neuromodulation systems can produce applicable acute or long-term effects. The latter occur through Long-Term Depression (LTD) or Long-Term Potentiation (LTP) via training. Included is control of direction of the energy emission, intensity, frequency (carrier frequency and/or neuromodulation frequency), pulse duration, pulse pattern, and phase/intensity relationships to targeting and accomplishing up-regulation and/or down-regulation.

The stimulation frequency for inhibition is lower than 400 Hz (depending on condition and patient). The stimulation frequency for excitation is in the range of 600 Hz to 4.5 MHz. In this invention, the ultrasound acoustic frequency is in range of 0.25 MHz to 0.85 MHz with power generally applied less than 65 mW/cm² but also at higher target- or patient-specific levels at which no tissue damage is caused. The acoustic frequency is modulated at the lower rate to impact the neuronal structures as desired (e.g., say typically 400 Hz for inhibition (down-regulation) or 600 Hz for excitation (up-regulation). The modulation frequency (superimposed on the carrier frequency of say 0.55 MHz or similar) may be divided into pulses 0.1 to 20 msec. repeated at frequencies of 2 Hz or lower for down regulation and higher than 2 Hz for up regulation) although this will be both patient and condition specific. The focus area of the pulsed ultrasound js 0.1 to 1 inch in diameter. The number of ultrasound transducers can vary between one and 100. Ultrasound therapy can be combined with therapy using other devices (e.g., Transcranial Magnetic Stimulation (TMS)).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 1 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

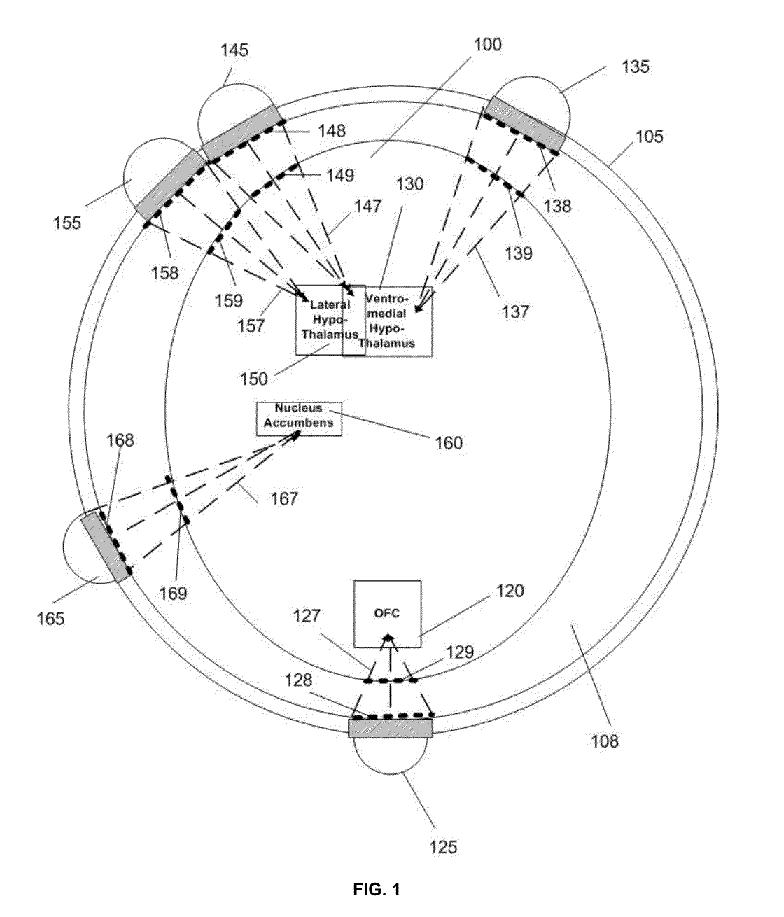

FIG. 1 shows a set of ultrasound transducers targeting to treat obesity. Head 100 contains the four targets, Orbito-Frontal Cortex (OFC) 120, Ventromedial Hypothalamus (VMH) 130, Lateral Hypothalamus (LH) 150, and Nucleus Accumbens 160. Note that while these three targets are covered here, fewer can work as well, or an addition or substitution of other targets identified in the future. These targets are hit by ultrasound from transducers 125, 135, 145, 155, and 165 fixed to track 105. Ultrasound transducer 125 with its beam 127 is shown targeting the Orbito-Frontal Cortex (OFC) 120 which would be down regulated, transducer 135 with its beam 137 is shown targeting the Ventromedial Hypothalamus (VMH) 130 (left side) and transducer 145 with its beam 147 also targeting the Ventromedial Hypothalamus 130 (right side) because the Ventromedial Thalamus would be down regulated bilaterally, transducer 155 with its beam 157 is shown targeting the Lateral Hypothalamus (LH) 150 that would also be down regulated, and transducer 165 with its beam 167 is shown targeting the Nucleus Accumbens 160 that is down regulated. For ultrasound to be effectively transmitted to and through the skull and to brain targets, coupling must be put into place. Ultrasound transmission (for example Dermasol from California Medical Innovations) medium 108 is interposed with one mechanical interface to the frame 105 and ultrasound transducers 125, 135, 145, 155, and 165 (completed by a layer of ultrasound transmission gel layers 128, 138, 148, 158, and 168) and the other mechanical interface to the head 100 (completed by a layers of ultrasound transmission gel 129, 139, 149, 159, and 169). In another embodiment the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target.

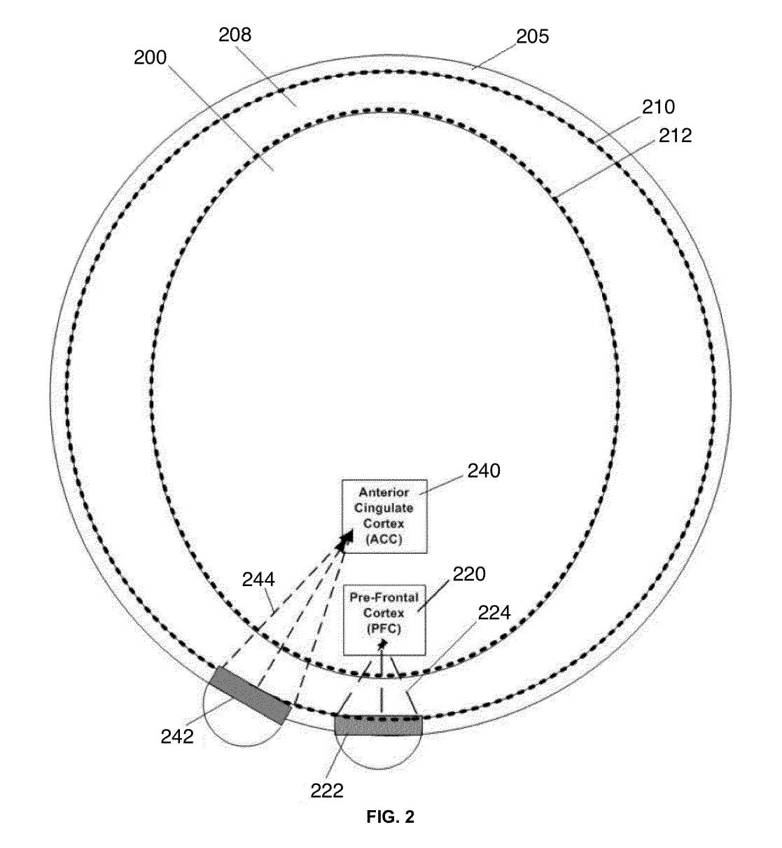

FIG. 2 shows a set of ultrasound transducers targeting to treat the eating disorder Anorexia Nervosa. Head 200 contains two targets, Pre-Frontal Cortex (PFC) 220 (to be down regulated) and Anterior Cingulate Cortex 240 (to be up regulated). While these two targets are covered here, others might work as well, or an addition or substitution of other targets (e.g., Posterior Cingulate Cortex (PCC), Right Dorso-Lateral Pre-Frontal Cortex (DLPFC), the Anterior Cingulate Cortex (ACC), and the Medial Pre-Frontal Cortex (MPFC) identified currently or in the future. The targets shown are hit by ultrasound from transducers 222 and 242 fixed to track 205. Ultrasound transducer 222 with its beam 224 is shown targeting Pre-Frontal Cortex (PFC) 220 and transducer 242 with its beam 244 is shown targeting Anterior Cingulate Cortex 240. For ultrasound to be effectively transmitted to and through the skull and to brain targets, coupling must be put into place. Ultrasound transmission (for example Dermasol from California Medical Innovations) medium 208 is interposed with one mechanical interface to the frame 205 and ultrasound transducers 222 and 242 (completed by a layer of ultrasound transmission gel layer 210) and the other mechanical interface to the head 200 (completed by a layer of ultrasound transmission gel 212). In another embodiment the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers.

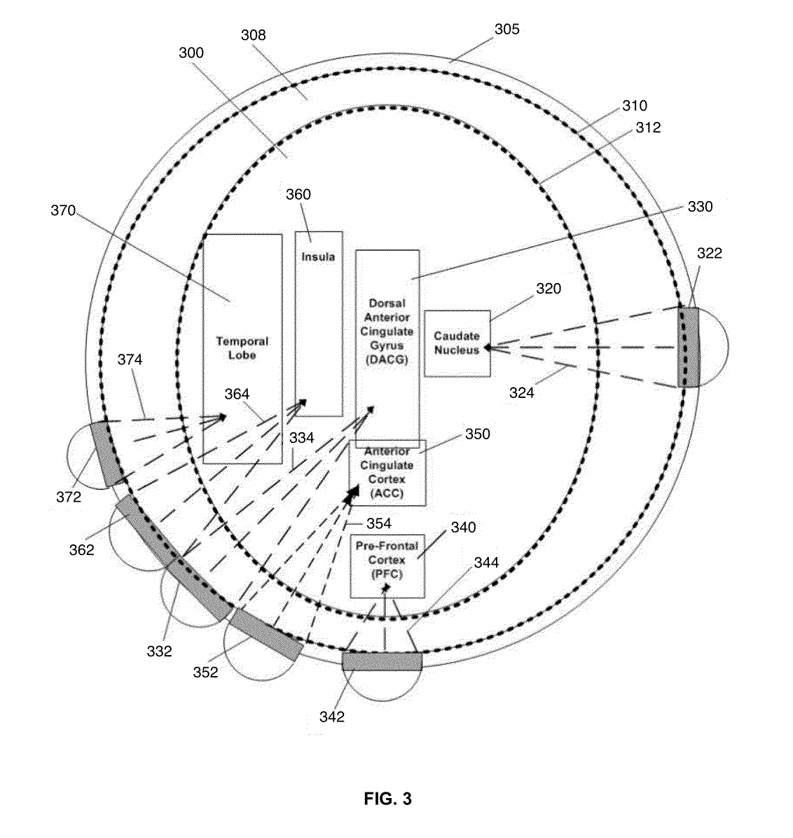

FIG. 3 shows a set of ultrasound transducers targeting to treat the eating disorder Bulimia. Head 300 contains six targets, Caudate Nucleus 320 (to be up regulated), the Dorsal Anterior Cingulate Gyms (DACG) 330 (to be down regulated), Pre-Frontal Cortex (PFC) 340 (to be up regulated), Anterior Cingulate Cortex (ACC) 350 (to be up regulated), the Insula 360 (to be up regulated), and Temporal Lobe 370 (to be up regulated). Note that while these six targets are covered here, others can work as well. The targets shown are hit by ultrasound from transducers 322, 332, 342, 352, 362, and 372 fixed to track 305. Ultrasound transducer 322 with its beam 324 is shown targeting Caudate Nucleus 320, transducer 332 with its beam 334 is shown targeting Dorsal Anterior Cingulate Gyms (DACG) 330, transducer 342 with its beam 344 is shown targeting Pre-Frontal Cortex (PFC) 340, transducer 352 with its beam 354 is shown targeting Anterior Cingulate Cortex (ACC) 350, transducer 362 with its beam 364 is shown targeting Insula 360, and transducer 372 with its beam 374 is shown targeting Temporal Lobe 370. Bilateral stimulation of one of a plurality of these targets is another embodiment. For ultrasound to be effectively transmitted to and through the skull and to brain targets, coupling must be put into place. Ultrasound transmission (for example Dermasol from California Medical Innovations) medium 308 is interposed with one mechanical interface to the frame 305 and ultrasound transducers 322, 332, 342, 352, 362, and 372 (completed by a layer of ultrasound transmission gel layer 310) and the other mechanical interface to the head 300 (completed by a layer of ultrasound transmission gel 312). In another embodiment the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers.

Transducer array assemblies of this type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—31/07—02/08/02), typically with numbers of ultrasound transducers of 300 or more. Keramos-Etalon in the U.S. is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

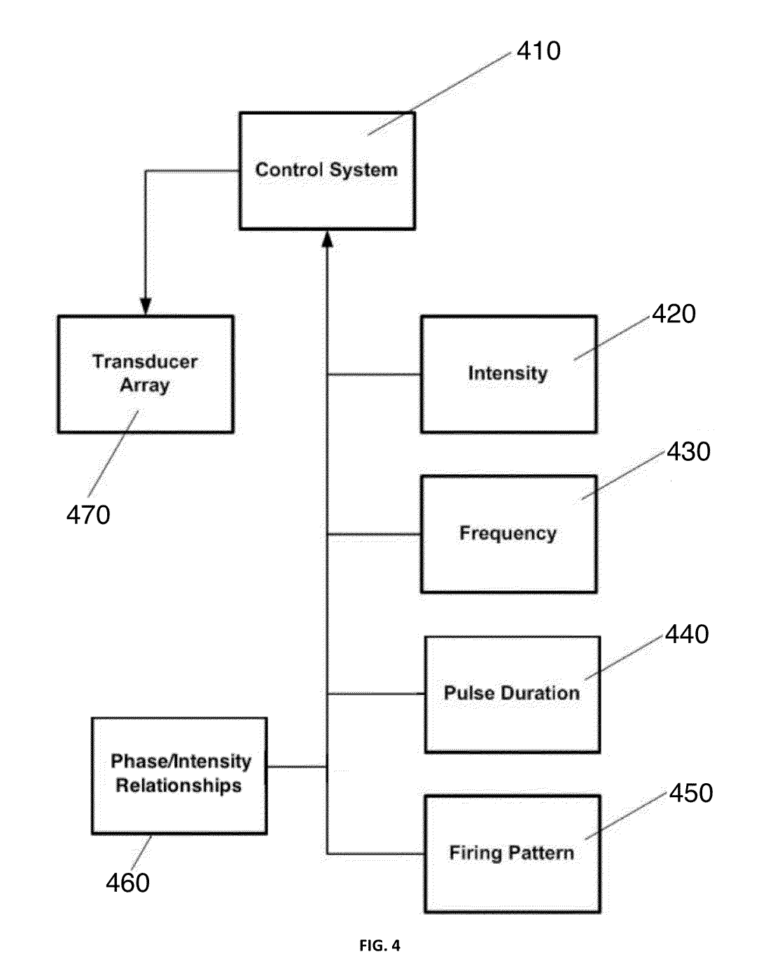

FIG. 4 shows an embodiment of a control circuit. The positioning and emission characteristics of transducer array 470 are controlled by control system 410 with control input with neuromodulation characteristics determined by settings of intensity 420, frequency 430, pulse duration 440, firing pattern 450, and phase/intensity relationships 460 for beam steering and focusing on neural targets.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

In still other embodiments, other energy sources are used in combination with or substituted for ultrasound transducers that are selected from the group consisting of Transcranial Magnetic Stimulation (TMS), deep-brain stimulation (DBS), optogenetics application, radiosurgery, Radio-Frequency (RF) therapy, behavioral therapy, and medications.

The invention allows stimulation adjustments in variables such as, but not limited to, direction of the energy emission, intensity, frequency (carrier frequency and/or neuromodulation frequency), pulse duration, pulse pattern, and phase/intensity relationships to targeting and accomplishing up-regulation and/or down-regulation, dynamic sweeps, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of deep-brain neuromodulation using ultrasound stimulation, the method comprising:
aiming one or a plurality of ultrasound transducer at one or a plurality of neural targets, and
applying pulsed power to the ultrasound transducer via a control circuit,
whereby the condition treated is selected from the group consisting of obesity and eating disorders.

2. The method of claim 1 further comprising aiming an ultrasound transducer neuromodulating neural targets in a manner selected from the group of up-regulation, down-regulation.

3. The method of claim 1 wherein the effect is chosen from the group consisting of acute, Long-Term Potentiation, and Long-Term Depression.

4. The method of claim 1 wherein one or a plurality of targets for the treatment of obesity are selected from the group consisting of Orbito-Frontal Cortex, Ventromedial Hypothalamus, Lateral Hypothalamus, and Nucleus Accumbens.

5. The method of claim 1 wherein the eating disorder is selected from the group selected from Anorexia Nervosa and Bulimia Nervosa.

6. The method of claim 1 wherein one or a plurality of targets for the treatment of eating disorders are selected from the group consisting of Caudate Nucleus, the Dorsal Anterior Cingulate Gyms (DACG), Pre-Frontal Cortex (PFC), Anterior Cingulate Cortex (ACC), the Insula, and Temporal Lobe.

7. The method of claim 1 wherein a single ultrasonic transducer aimed at a given target is replaced by a plurality of ultrasonic transducers whose beams intersect at that target.

8. The method of claim 1 wherein the acoustic ultrasound frequency is in the range of 0.25 MHz to 0.85 MHz.

9. The method of claim 1 where in the power applied is less than 65 mW/cm².

10. The method of claim 1 wherein the power applied is greater than 65 mW/cm² but less than that causing tissue damage.

11. The method of claim 1 wherein a stimulation frequency of lower than 400 Hz is applied for inhibition of neural activity.

12. The method of claim 10 wherein modulation frequency of lower than 400 Hz is divided into pulses 0.1 to 20 msec. repeated at frequencies of 2 Hz or lower for down regulation.

13. The method of claim 1 wherein the stimulation frequency for excitation is in the range of 600 Hz to 4.5 MHz.

14. The method of claim 12 wherein modulation frequency of 600 Hz or higher is divided into pulses 0.1 to 20 msec. repeated at frequencies higher than 2 Hz for up regulation.

15. The method of claim 1 wherein the focus area of the pulsed ultrasound is 0.1 to 1 inch in diameter.

16. The method of claim 1 wherein the number of ultrasound transducers is between 1 and 100.

17. The method of claim 1 wherein mechanical perturbations are applied radially or axially to move the ultrasound transducers.

18. The method of claim 1 wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

19. The method of claim 1 wherein ultrasound therapy is combined with or replaced by one or more therapies selected from the group consisting of Transcranial Magnetic Stimulation (TMS), deep-brain stimulation (DBS), application of optogenetics, radiosurgery, Radio-Frequency (RF) therapy, behavioral therapy, and medications.
